(19) United States
(12) Patent Application Publication
(10) Pub. No.: US 2007/0082027 A1
(43) Pub. Date: Apr. 12, 2007

(54) Compositions and methods for reducing food intake and controlling weight

(76) Inventors: William Ronald Aimutis JR., Blaine, MN (US); Steven J. Catani, Athens, GA (US); Eugene Terry Finocchiaro, West Amwell, NJ (US); Teresa Marie Paeschke, Minneapolis, MN (US); Thomas E. Sox, Ambler, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/245,872

(22) Filed: Oct. 7, 2005

(51) Int. Cl. A61K 47/00

(52) U.S. Cl. 424/439

(57) ABSTRACT

An ingestible composition to increase viscosity imparted to the digesta in an intestine that includes a soluble anionic fiber and a protected cation. The protected cation may be coated with an enteric coating, a lipid coating, or a sustained release coating.

### CROSS-REFERENCE TO RELATED APPLICATIONS

This case is related to U.S. patent application Ser. No. ______, entitled “COMPOSITIONS AND METHODS FOR REDUCING FOOD INTAKE AND CONTROLLING WEIGHT” (docket number MSP5038); U.S. patent application Ser. No ______, entitled “METHODS FOR REDUCING CALORIE INTAKE” (docket number MSP5039), U.S. patent application Ser. No. ______, entitled “COMPOSITIONS AND METHODS FOR INDUCING SATIETY AND REDUCING CALORIC INTAKE” (docket number MSP5040); U.S. patent application Ser. No. ______, entitled “METHODS FOR ACHIEVING AND MAINTAINING WEIGHT LOSS” (docket number MSP5041); U.S. patent application Ser. No. ______, entitled “METHODS FOR REDUCING WEIGHT” (docket number MSP5042); U.S. patent application Ser. No. ______, entitled “COMPOSITIONS AND METHODS FOR REDUCING FOOD INTAKE AND CONTROLLING WEIGHT” (docket number MSP5044); U.S. patent application Ser. No. ______, entitled “METHODS FOR WEIGHT MANAGEMENT” (docket number MSP5045); U.S. patent application Ser. No. ______, entitled “METHODS FOR INDUCING SATIETY, REDUCING FOOD INTAKE AND REDUCING WEIGHT” (docket number MSP5046); U.S. patent application Ser. No. ______, entitled “COMPOSITIONS AND METHODS FOR REDUCING FOOD INTAKE AND CONTROLLING WEIGHT (docket number MSP5047); U.S. patent application Ser. No. ______, entitled “FIBER SATIETY COMPOSITIONS” (docket number 10790-7334-1 056001); and U.S. patent application Ser. No. ______, entitled “FIBER SATIETY COMPOSITIONS” (docket number 10790-056002), each filed concurrently herewith on Oct. 7, 2005.

### FIELD OF THE INVENTION

The present invention is directed to ingestible compositions that include at least one anionic soluble fiber and at least one protected cation, methods for making the ingestible compositions, and methods of using the ingestible compositions to increase viscosity imparted to the digesta in an intestine.

### BACKGROUND OF THE INVENTION

Diabetes and obesity are common ailments in the United States and other Western cultures. A study by researchers at RTI International and the Centers for Disease Control estimated that U.S. obesity-attributable medical expenditures reached $75 billion in 2003. Obesity has been shown to promote many chronic diseases, including type 2 diabetes, cardiovascular disease, several types of cancer, and gallbladder disease.

Adequate dietary intake of soluble fiber has been associated with a number of health benefits, including decreased blood cholesterol levels, improved glycemic control, and the induction of satiety and satiation in individuals. Consumers have been resistant to increasing soluble fiber amounts in their diet, however, often due to the negative organoleptic characteristics, such as, sliminess, excessive viscosity, and poor flavor, that are associated with food products that include soluble fiber.

However, viscosity in the stomach delays gastric emptying, which delays the time it takes to get signaling agents to the ileum to cause satiety. This results in food intake reduction. There is a need to delay viscosity formation until the digesta reaches the intestine because the speed and efficient delivery of signaling agents to the ileum can be achieved simultaneously.

### SUMMARY OF THE INVENTION

The present invention solves those and other needs. One embodiment of the present invention is an ingestible composition to increase viscosity imparted to the digesta in an intestine the composition comprising, consisting of, and/or consisting essentially of an effective amount of at least one soluble anionic fiber and an effective amount of at least one protected cation to increase viscosity imparted to the digesta in an intestine.

Another embodiment of the present invention is an ingestible composition comprising, consisting of, and/or consisting essentially of at least one anionic soluble fiber and a protected neutral pH soluble calcium salt in solid form.

A further embodiment of the present invention is an ingestible composition comprising, consisting of, and/or consisting essentially of: at least one anionic soluble fiber and a cation containing lipid composition, the cation- containing lipid composition comprising, consisting of, and/or consisting essentially of a neutral pH soluble multivalent cation salt or complex, wherein the multivalent cation salt or complex is at least partially coated with a composition of monoglycerides, diglycerides, or triglycerides, or combinations thereof.

A still further embodiment of the present invention is an ingestible composition comprising, consisting of, and/or consisting essentially of at least one anionic soluble fiber and a plurality of enteric coated cations.

### BRIEF DESCRIPTION OF DRAWINGS

FIG. 1 is a linear graph of the effects of enteric coated and uncoated calcium on viscosity of intestinal contents.

FIG. 2 is a linear graph of calcium dissolution of lipid coated particles at pH 2.5.

FIG. 3 is a linear graph of calcium dissolution of lipid coated particles at pH 7.2.

### DETAILED DESCRIPTION OF THE INVENTION

As used herein, unless indicated otherwise, the terms “alginate,” “pectin,” “carrageenan,” “polygeenan,” or “gellan” refers to all forms (e.g., protonated or salt forms, such as sodium, potassium, and ammonium salt forms and having varying average molecular weight ranges) of the anionic soluble fiber type.

As used herein, unless indicated otherwise, the term “alginic acid” includes not only the material in protonated form but also the related salts of alginate, including but not limited to sodium, potassium, and ammonium alginate.

As used herein, unless indicated otherwise, the term “protected” means that the cation material has been treated in such a way as to delay (e.g., until during or after ingestion or until a certain pH range has been reached) reaction of the at least one protected cation with the anionic soluble fiber as compared to an unprotected cation.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, a recitation of a range of values is merely intended to serve as a shorthand method of referring individually to each separate value falling within the range, and each separate value is incorporated into the specification as if it were individually recited herein.

The compositions of this invention reduce food intake. The inventors believe that this arises from the enhanced viscosity produced by the interactions of cations and a soluble anionic fiber.

Soluble Anionic Fiber

Any soluble anionic fiber should be acceptable for the purposes of this invention. Suitable soluble anionic fibers include alginate, pectin, gellan, soluble fibers that contain carboxylate substituents, carrageenan, polygeenan, and marine algae-derived polymers that contain sulfate substituents.

Also included within the scope of soluble anionic fibers are other plant derived and synthetic or semisynthetic polymers that contain sufficient carboxylate, sulfate, or other anionic moieties to undergo gelling in the presence of sufficient levels of cation.

At least one source of soluble anionic fiber may be used in these compositions, and the at least one source of soluble anionic fiber may be combined with at least one source of soluble fiber that is uncharged at neutral pH. Thus, in certain cases, two or more anionic soluble fibers types are included, such as, alginate and pectin, alginate and gellan, or pectin and gellan. In other cases, only one type of anionic soluble fiber is used, such as only alginate, only pectin, only carrageenan, or only gellan.

Anionic soluble fibers are commercially available, e.g., from ISP (Wayne, N.J.), TIC Gums, and CP Kelco.

An alginate can be a high guluronic acid alginate. For example, in certain cases, an alginate can exhibit a higher than 1:1 ratio of guluronic to mannuronic acids, such as in the range from about 1.2:1 to about 1.8:1, e.g., about 1.3:1, about 1.4:1, about 1.5:1, about 1.6:1, or about 1.7:1 or any value therebetween. Examples of high guluronic alginates (e.g., having a higher than 1:1 g:m ratios) include Manugel LBA, Manugel GHB, and Manugel DBP, which each have a g:m ratio of about 1.5.

While not being bound by theory, it is believed that high guluronic alginates can cross-link through cations, e.g., calcium ions, to form gels at the low pH regimes in the stomach. High guluronic alginates are also believed to electrostatically associate with pectins and/or gellans at low pHs, leading to gellation. In such cases, it may be useful to delay the introduction of cations until after formation of the mixed alginate/pectin or alginate/gellan gel, as cationic cross-links may stabilize the mixed gel after formation.

In other cases, an alginate can exhibit a ratio of guluronic to mannuronic acids (g:m ratio) of less than 1:1, e.g., 0.8:1 to about 0.4:1, such as about 0.5:1, about 0.6: 1, or about 0.7:1 or any value therebetween. Keltone LV and Keltone HV are examples of high-mannuronic acids (e.g., having a g:m ratio of less than 1:1) having g:m ratios ranging from about 0.6:1 to about 0.7:1.

Methods for measuring the ratio of guluronic acids to mannuronic acids are known by those having ordinary skill in the art.

An alginate can exhibit any number average molecular weight range, such as a high molecular weight range (about 2.05×10⁵ to about 3×10⁵ Daltons or any value therebetween; examples include Manugel DPB, Keltone HV, and TIC 900 Alginate); a medium molecular weight range (about 1.38×10⁵ to about 2×10⁵ Daltons or any value therebetween; examples include Manugel GHB); or a low molecular weight range (about 2×10⁴ to about 1.35×10⁵ Daltons or any value therebetween; examples include Manugel LBA and Manugel LBB). Number average molecular weights can be determined by those having ordinary skill in the art, e.g., using size exclusion chromatography (SEC) combined with refractive index (RI) and multi-angle laser light scattering (MALLS).

In certain embodiments of an extruded food product, a low molecular weight alginate can be used (e.g., Manugel LBA), while in other cases a mixture of low molecular weight (e.g., Manugel LBA) and high molecular weight (e.g., Manugel DPB, Keltone HV) alginates can be used. In other cases, a mixture of low molecular weight (e.g., Manugel LBA) and medium molecular weight (e.g., Manugel GHB) alginates can be used. In yet other cases, one or more high molecular weight alginates can be used (e.g., Keltone HV, Manugel DPB).

A pectin can be a high-methoxy pectin (e.g., having greater than 50% esterified carboxylates), such as ISP HM70LV and CPKelco USPL200. A pectin can exhibit any average molecular weight range, including a low molecular weight range (about 1×10⁵ to about 1.20×10⁵ Daltons, e.g., CP Kelco USPL200), medium molecular weight range (about 1.25×10⁵ to about 1.45×10⁵, e.g., ISP HM70LV), or high molecular weight range (about 1.50×10⁵ to about 1.80×10⁵, e.g., TIC HM Pectin). In certain cases, a high-methoxy pectin can be obtained from pulp, e.g., as a by-product of orange juice processing.

A gellan anionic soluble fiber can also be used. Gellan fibers form strong gels at lower concentrations than alginates and/or pectins, and can cross-link with mono- and multivalent cations. For example, gellan can form gels with sodium, potassium, magnesium, and calcium. Gellans for use in the invention include Kelcogel, available commercially from CP Kelco.

Fiber blends as described herein can also be used in the preparation of a solid ingestible composition like an extruded food product where the fiber blend is a source of the soluble anionic fiber. A useful fiber blend can include an alginate soluble anionic fiber and a pectin soluble anionic fiber. A ratio of total alginate to total pectin in a blend can be from about 8:1 to about 5:1, or any value therebetween, such as about 7:1, about 6.5:1, about 6.2:1, or about 6.15:1. A ratio of a medium molecular weight alginate to a low molecular weight alginate can range from about 0.65:1 to about 2:1, or any value therebetween.

An alginate soluble anionic fiber in a blend can be a mixture of two or more alginate forms, e.g., a medium and low molecular weight alginate. In certain cases, a ratio of a medium molecular weight alginate to a low molecular weight alginate is about 0.8:1 to about 0.9:1.

The at least one anionic soluble fiber may be treated before, during, or after incorporation into an ingestible composition. For example, the at least one anionic soluble fiber can be processed, e.g., extruded, roll-dried, freeze-dried, dry blended, roll-blended, agglomerated, coated, or spray-dried.

For solid forms, a variety of extruded shapes of food products can be prepared by methods known to those having ordinary skill in the art, e.g., extruding, molding, pressing, wire cutting, and the like. For example, a single or double screw extruder can be used. Typically, a feeder meters in the raw ingredients to a barrel that includes the screw(s). The screw(s) conveys the raw material through the die that shapes the final product. Extrusion can take place under high temperatures and pressures or can be a non-cooking, forming process. Extruders are commercially available, e.g., from Buhler, Germany. Extrusion can be cold or hot extrusion.

Other processing methods are known to those having skilled in the art.

The amount of the at least one anionic soluble fiber included can vary, and will depend on the type of ingestible composition and the type of anionic soluble fiber used. For example, typically a solid ingestible composition will include from about 0.5 g to about 10 g total soluble anionic fiber per serving or any value therebetween. A preferred range of fiber intake in the compositions of this invention is about 0.25 g to 5 g per serving, more preferably about 0.5 to 3 g per serving, and most preferably about 1.0 to 2.0 g per serving. In certain cases, an extruded food product can include an anionic soluble fiber at a total amount from about 22% to about 40% by weight of the extruded product or any value therebetween. In other cases, an extruded food product can include an anionic soluble fiber in a total amount of from about 4% to about 15% or any value therebetween, such as when only gellan is used. In yet other cases, an extruded food product can include an anionic soluble fiber at a total amount of from about 18% to about 25% by weight, for example, when combinations of gellan and alginate or gellan and pectin are used.

In addition to the at least one anionic soluble fiber, a solid ingestible composition can include ingredients that may be treated in a similar manner as the at least one anionic soluble fiber. For example, such ingredient can be co-extruded with the anionic soluble fiber, co-processed with the anionic soluble fiber, or co-spray-dried with the anionic soluble fiber. Such treatment can help to reduce sliminess of the ingestible composition in the mouth and to aid in hydration and gellation of the fibers in the stomach and/or small intestine. Without being bound by any theory, it is believed that co-treatment of the anionic soluble fiber(s) with such ingredient prevents early gellation and hydration of the fibers in the mouth, leading to sliminess and unpalatability. In addition, co-treatment may delay hydration and subsequent gellation of the anionic soluble fibers (either with other anionic soluble fibers or with cations) until the ingestible composition reaches the stomach and/or small intestine, providing for the induction of satiety and/or satiation.

Additional ingredients can be hydrophilic in nature, such as starch, protein, maltodextrin, and inulin. Other additional ingredients can be insoluble in water (e.g., cocoa solids, corn fiber) and/or fat soluble (vegetable oil), or can be flavor modifiers such as sucralose. For example, an extruded food product can include from about 5 to about 80% of a cereal ingredient, such as about 40% to about 68% of a cereal ingredient. A cereal ingredient can be rice, corn, wheat, sorghum, oat, or barley grains, flours, or meals. Thus, an extruded food product can include about 40% to about 50%, about 50% to about 58%, about 52% to about 57%, or about 52%, 53%, 54%, 55%, 56%, or 56.5% of a cereal ingredient. In one embodiment, about 56.5% of rice flour is included.

An ingestible composition can also include a protein source. A protein source can be included in the composition or in an extruded food product. For example, an extruded food product can include a protein source at about 2% to about 20% by weight, such as about 3% to about 8%, about .3% to about 5%, about 4% to about 7%, about 4% to about 6%, about 5% to about 7%, about 5% to about 15%, about 10% to about 18%, about 15% to about 20%, or about 8% to about 18% by weight. A protein can be any known to those having ordinary skill in the art, e.g., rice, milk, egg, wheat, whey, soy, gluten, or soy flour. In some cases, a protein source can be a concentrate or isolate form.

Protected Cation

The compositions and associated methods of this invention include at least one protected cation in an amount sufficient to cause an increase in viscosity of the anionic soluble fiber. The at least one protected cation may be incorporated into an ingestible composition provided herein, or can be consumed as a separate food article either before, after, or simultaneously with an ingestible composition.

A protected cation can be a monovalent or multivalent (or polyvalent) cation. Cations useful in this invention include potassium, sodium, calcium, magnesium, aluminum, manganese, iron, nickel, copper, zinc, strontium, barium, bismuth, chromium, vanadium, and lanthanum, their salts and mixtures thereof. Salts of the cations may be organic acid salts that include formate, fumarate, acetate, propionate, butyrate, caprylate, valerate, lactate, citrate, malate and gluconate. Also included are highly soluble inorganic salts such as chlorides or other halide salts.

In certain compositions, one or more particular cations may be used with certain anionic soluble fibers, depending on the composition and gel strength desired. For example, for ingestible alginate compositions, calcium may be used to promote gellation. For gellan compositions, one or more of calcium, sodium, potassium, and magnesium may be used.

Typically, a separate food article containing the source of at least one protected cation would be consumed in an about four hour time window flanking the ingestion of an ingestible composition containing the at least one anionic soluble fiber. In certain cases, the window may be about three hours, or about two hours, or about one hour. In other cases, the separate food article may be consumed immediately before or immediately after ingestion of an ingestible composition, e.g., within about fifteen minutes, such as within about 10 minutes, about 5 minutes, or about 2 minutes. In other cases, a separate food article containing at least one protected cation can be ingested simultaneously with an ingestible composition containing the at least one soluble anionic fiber, e.g., a snack chip composition where some chips include at least one protected cation and some chips include the at least one soluble anionic fiber.

While not wishing to be bound to any particular theory, the inventors hypothesize that the calcium was released from the particles after the particles reached the neutral pH of the intestine, and the free calcium cross-linked some of the carboxylate groups on the alginate polymers. This crossing created a more rigid matrix of greater apparent molecular weight, which increased the viscosity of the samples in comparison to the water controls or the diluted beverage without calcium.

In one embodiment, at least one protect cation can be included in an ingestible composition in a different food matrix from a matrix containing an anionic soluble fiber. For example, a source of at least one protected cation, such as a protected calcium salt, can be included in a separate matrix of a solid ingestible composition from the matrix containing the at least one soluble anionic fibers. Thus, means for physical separation of an anionic soluble fiber (e.g., within a snack bar or other extruded food product) from a source of at least one protected cation are also contemplated, such as by including the source of at least one protected cation in a matrix such as a frosting, coating, drizzle, chip, chunk, swirl, or interior layer. In one embodiment, a source of at least one protected cation, such as a protected cation source, can be included in a snack bar matrix that also contains an extruded crispy matrix that contains the anionic soluble fiber. In such a case, the source of at least one protected cation is in a separate matrix than the extruded crispy matrix containing the anionic soluble fiber. In another embodiment, a source of at least one protected cation can be included in a gel layer, e.g., a jelly or jam layer.

One cation source is cation salts. Typically, a cation salt can be selected from the following salts: citrate, tartrate, malate, formate, lactate, gluconate, phosphate, carbonate, sulfate, chloride, acetate, propionate, butyrate, caprylate, valerate, fumarate, adipate, and succinate. In certain cases, a cation salt is a calcium salt. A calcium salt can have a solubility of >1% w/vol in water at pH 7 at 20° C. A calcium salt can be, without limitation, calcium citrate, calcium tartrate, calcium malate, calcium lactate, calcium gluconate, calcium citrate malate, dicalcium phosphate dihydrate, anhydrous calcium diphosphate, dicalcium phosphate anhydrous, tricalcium phosphate, calcium carbonate, calcium sulfate dihydrate, calcium sulfate anhydrous, calcium chloride, calcium acetate monohydrate, monocalcium phosphate monohydrate, and monocalcium phosphate anhydrous.

The diameter and geometry of the cation may provide additional control on the rate of passage from the stomach to the intestine. Material passing from the stomach through the pyloric sphincter into the duodenum is subjected to a sieving procedure. Early in the digestion of a meal, the slit-like pylorus opens only slightly, allowing liquid and small particles to exit, but retaining larger particles for further degradation by enzymatic attack, stomach acid and antral grinding. Later in the digestion of a meal the pylorus opens wider so that larger particles can exit, and late in the digestive process a “dumping wave” sweeps through the stomach, allowing removal of coarse non-digestible particulate matter. Soluble anionic fiber is able to leave the stomach early in the digestion process. Similarly, particles of about 3 mm diameter or less are also able to exit the human stomach early in the digestion process. An important aspect of this invention is the contemporaneous presence of fiber and cation in the small intestine. Therefore, a preferred embodiment of this invention is the use of cations that less than about 5 mm diameter, more preferably about 3 mm or less in diameter, and most preferably about 100 microns to about 3 mm in diameter.

A number of methods can be used to protect the cation of the present invention. The cation can be coated with an enteric or enteric-like coating, a sustained release coating, lipid coating and the like. In addition, microparticles or nanoparticles having double or multiple emulsions, such as water/oil/water (“w/o/w”) or oil/water/oil (“o/w/o”) emulsions, of at least one protected cation and an anionic soluble fiber can be used. In one embodiment, a calcium alginate microparticle or nanoparticle is used.

As particle size decreases, the relative surface area per mass of particle increases. For particles that are subjected to an enteric or enteric-like coating, this results in the coating comprising a larger portion of the mass of the final particles. This increases cost and decreases the amount of multivalent cation delivered for a given mass. Therefore, although small particles (e.g., less than about 10 to about 100 μM in diameter) are advantageous for coated cations from organoleptic sensory and product formulation standpoints, the relatively high amount of coating used decreases the amount of cation delivered on a weight basis.

Calcium chloride solution can be emulsified in oil, which emulsion can then be dispersed in a continuous water phase containing the anionic alginate soluble fiber. When the emulsion breaks in the stomach, the calcium can react with the alginate to form a gel.

A microparticle can have a size from about 1 to about 15 μM (e.g., about 5 to about 10 μM, or about 3 to about 8 μM). A nanoparticle can have a size of about 11 to about 85 nm (e.g., about 15 to about 50 nm, about 30 to about 80 nm, or about 50 to about 75 nm). The preparation of multiple or double emulsions, including the choice of surfactants and lipids, is known to those having ordinary skill in the art.

In another embodiment, nanoparticles of calcium alginate are formed by preparing nanodroplet w/o microemulsions of CaCl₂ in a solvent and nanodroplet w/o microemulsions of alginate in the same solvent. When the two microemulsions are mixed, nanoparticles of calcium alginate are formed. The particles can be collected and dispersed, e.g., in a liquid ingestible composition. As the particle size is small (<100 nm), the particles stay dispersed (e.g., by Brownian motion), or can be stabilized with a food grade surfactant. Upon ingestion, the particles aggregate and gel.

In other embodiments, a liposome containing a source of at least one protected cation can be included in an ingestible composition. For example, a calcium-containing liposome can be used. The preparation of liposomes containing cations is well known to those having ordinary skill in the art; see ACS Symposium Series, 1998 709:203-211; Chem. Mater. 1998 (109-116). Cochelates can also be used, e.g., as described in U.S. Pat. No. 6,592,894 and U.S. Pat. No. 6,153, 217. The creation of cochelates using cations such as calcium can protect the cations from reacting with the anionic soluble fiber within the aqueous phase of an ingestible composition, e.g., by wrapping the cations in a hydrophobic lipid layer, thus delaying reaction with the fiber until digestion of the protective lipids in the stomach and/or small intestine via the action of lipases.

In certain cases, a cation-containing carbohydrate glass can be used, such as a calcium containing carbohydrate glass. A carbohydrate glass can be formed from any carbohydrate such as, without limitation, sucrose, trehalose, inulin, maltodextrin, corn syrup, fructose, dextrose, and other mono-, di-, or oligo-saccharides using methods known to those having ordinary skill in the art; see, e.g., WO 02/05667. A carbohydrate glass can be used, e.g., in a coating or within a food matrix.

The amount of protected cation required will be proportional to the level of anionic fiber. Generally the amount of protected cation needed (not considering the mass of the counterion or complexing agent) will be about 3% to about 20% weight percent of the weight of soluble anionic fiber, more preferably about 6% to 15% weight percent.

Coating

The enteric solubilization of the protected cation particles is required so that substantial solubilization occurs in the small intestine, thereby promoting the increased viscosity or gelling of the soluble anionic fiber that is ingested simultaneously with, or within approximately two hours before or after the ingestion of the anionic fiber. A variety of methods and compositions may be used to achieve enteric solubilization of the multivalent cation particles.

One method is coating the cation with an enteric coating. The enteric coat substantially prevents the solubilization of the cation in the stomach. The cation particles may be coated to provide for prolonged release or delayed pulse solubilization of the cation, or they may be designed to provide for a combination of immediate, pulsed and/or prolonged delivery of cation. In addition, there may be formulations that contain more than one type of cation.

The enteric coating composition can be a member selected from the group consisting of cellulose acetyl phthalate, cellulose diacetyl phthalate, cellulose triacetyl phthalate, cellulose acetate phthalate, hydroxypropylmethylcellulose phthalate, sodium cellulose acetate phthalate, cellulose ester phthalate, cellulose ether phthalate, methylcellulose phthalate, cellulose ester-ether phthalate, hydroxypropyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate diethyl phthalate, dibutyl phthalate, dialkyl phthalate wherein the alkyl comprises from 1 to 7 straight and branched alkyl groups, aryl phthalates, and the like. The cation can also be coated with a suitable enteric coating polymer; non-limiting examples of these are Eudragit materials (Rohm Pharma), and Acryl EZE MP (Colorcon, Inc, West Point, Pa.).

The particles may also contain agents that slow the dissolution of the multivalent cation source after dissolution of the enteric coating. Non-limiting examples of these materials are methylcellulose, ethylcellulose, propylcellulose, and starch or starch derivatives.

This invention also envisions the use enteric polymers other than the materials traditionally used for enteric coatings in the pharmaceutical industry. These include modified or substituted starches, chitosan, and hydrophobic proteins such as maize zein.

Additionally, the compression or compaction force used in manufacture of an uncoated cation may also be used to control the intestinal dissolution rates.

The inventors have also discovered that release properties similar to those obtained with classical enteric coatings can be achieved using lipid matrices. These particles are formed by providing the cation of an appropriate particle size and applying a molten lipid coating to the particles. The lipid coating serves to agglomerate and coat the cation particles. The appropriately formulated particles show very effective enteric release properties. That is, there is minimal solubilization of the multivalent cation under conditions of gastric acidity. However, when the pH is increased to approximately neutral by the addition of suitable buffer, there is a rapid release of the cation. Although not wishing to be bound by theory, the inventors hypothesize that this behavior arises from conformational changes in the lipids or the surfaces properties of the lipid-coated cation, as well as enhanced solubility of cation salts under more neutral conditions. Additionally, the presence of pancreatic lipase and other enzymes in the intestine degrades the lipids into fatty acids and monoglycerides, eroding the particles and allowing release of the multivalent cations. The presence of bile acids and bile salts in the intestine may additionally aid in the disruption of these particles.

Useful amounts of fiber and cation include a weight ratio of from about 5:1 to about 40:1, from about 7:1 to about 20:1, or from about 10:1 to about 14:1 or any value therebetween.

The inventors also envision that other sustained release matrices can be used to achieve intestinal release of effective amounts of multivalent cations. Particles formed from a cation source and one of the protecting agents mentioned above (e.g., modified celluloses) or other materials known in the pharmaceutical art such as modified starches may be used to achieve primarily intestinal release. One example is the OROS technology developed by the Alza Corporation (for example, see U.S. Pat. No. 4,765,989). This provides an ion pump mechanism that provides controlled intestinal release of materials. Intestine-specific release can be achieved by applying an enteric coating to the entire OROS dosage form, or an enteric coating limited to the pore or pores that connect the dosage form contents to the external environment

Ingestible Compositions

Compositions of the present invention can be in any form, fluid or solid. Fluids can be beverages, including shake, liquado, and smoothie. Fluids can be from low to high viscosity.

Solid forms can extruded or not. Solid forms include bread, cracker, bar, cookie, confectioneries, e.g., nougats, toffees, caramels, hard candy enrobed soft core, muffins, cookies, brownies, cereals, chips, snack foods, bagels, chews, crispies, and nougats, pudding, jelly, and jam. Solids can have densities from low to high.

Fluids

Fluid ingestible compositions can be useful for, among other things, aiding in weight loss programs, e.g., as meal replacement beverages or diet drinks. Fluid ingestible compositions can provide from about 0.5 g to about 10 g of anionic soluble fiber per serving, or any value therebetween. For example, in certain cases, about 1 g, 2 g, 3 g, 4 g, 5 g, 6 g, 7 g, 8 g, or 9 g of at least one anionic soluble fiber are provided per serving.

A fluid ingestible composition may include an alginate anionic soluble fiber and/or a pectin anionic soluble fiber. In certain cases, an alginate anionic soluble fiber and a pectin anionic soluble fiber are used. A fiber blend as described herein can be used to provide the alginate anionic soluble fiber and/or the pectin anionic soluble fiber. An alginate and pectin can be any type and in any form, as described previously. For example, an alginate can be a high, medium, or low molecular weight range alginate, and a pectin can be a high-methoxy pectin. Also as indicated previously, two or more alginate forms can be used, such as a high molecular weight and a low molecular weight alginate, or two high molecular weight alginates, or two low molecular weight alginates, or a low and a medium molecular weight alginate, etc. For example, Manugel GHB alginate and/or Manugel LBA alginate can be used. In other cases, Manugel DPB can be used. Genu Pectin, USPL200 (a high-methoxy pectin) can be used as a pectin. In certain cases, potassium salt forms of an anionic soluble fiber can be used, e.g., to reduce the sodium content of an ingestible composition.

A fluid ingestible composition includes alginate and/or pectin in a total amount of about 0.3% to about 5% by weight, or any value therebetween, e.g., about 1.25% to about 1.9%; about 1.4% to about 1.8%; about 1.0% to about 2.2%, about 2.0% to about 4.0%, about 3.0%, about 4.0%, about 2.0%, about 1.5%, or about 1.5% to about 1.7%. Such percentages of total alginate and pectin can yield about 2 g to about 8 g of fiber per 8 oz. serving, e.g., about 3 g, about 4 g, about 5 g, about 6 g, or about 7 g fiber per 8 oz. serving. In other cases, about 4 g to about 8 g of fiber (e.g., about 5 g, about 6 g, or about 7 g) per 12 oz. serving can be targeted. In some embodiments, about 1.7% fiber by weight of a fluid ingestible composition is targeted.

In some cases, a fluid ingestible composition includes only alginate as a soluble anionic fiber. In other cases, alginate and pectin are used. A ratio of alginate to pectin (e.g., total alginate to total pectin) in a fluid ingestible composition can range from about 8:1 to about 1:8, and any ratio therebetween (e.g., alginate:pectin can be in a ratio of about 1:1, about 1.2:1, about 1.3:1, about 1.4:1, about 1.5:1, about 1.6:1, about 1.62:1, about 1.7:1, about 1.8:1, about 1.9:1, about 2:1, about 3:1, about 4:1, about 5:1, about 5.3:1, about 5.6:1, about 5.7:1, about 5.8:1, about 5.9:1, about 6:1, about 6.1:1, about 6.5:1, about 7:1, about 7.5:1, about 7.8:1, about 2:3, about 1:4, or about 0.88:1). In cases where alginate and pectin are in a ratio of about 0.5:1 to about 2:1, it is believed that pectin and alginate electrostatically associate with one another to gel in the absence of cations; thus, while not being bound by theory, it may be useful to delay the introduction of cations until after such gel formation. In other cases, where the ratio of alginate to pectin is in the range from about 3:1 to about 8:1, it may be useful to include a cation source such as a calcium source (e.g., to crosslink the excess alginate) to aid gel formation in the stomach. In these cases, the inventors believe, while not being bound by any theory, that the lower amount of pectin protects the alginate from precipitating as alginate at the low pHs of the stomach environment, while the cation source cross-links and stabilizes the gels formed.

A fluid ingestible-composition can have a pH from about 3.9 to about 4.5, e.g., about 4.0 to about 4.3 or about 4.1 to about 4.2. At these pHs, it is believed that the fluid ingestible compositions are above the pKas of the alginate and pectin acidic subunits, minimizing precipitation, separation, and viscosity of the solutions. In some cases, malic, phosphoric, and citric acids can be used to acidify the compositions. In some cases, a fluid ingestible composition can have a pH of from about 5 to about 7.5. Such fluid ingestible compositions can use pH buffers known to those having ordinary skill in the art.

Sweeteners for use in a fluid ingestible composition can vary according to the use of the composition. For beverages, low glycemic sweeteners may be preferred, including trehalose, isomaltulose, aspartame, saccharine, and sucralose. Sucralose can be used alone in certain formulations. The choice of sweetener will impact the overall caloric content of a fluid ingestible composition. In certain cases, a fluid ingestible compositions can be targeted to have 40 calories/12 oz serving.

A fluid ingestible composition can demonstrate gel strengths of about 20 to about 250 grams force (e.g., about 60 to about 240, about 150 to about 240, about 20 to 30, about 20 to about 55, about 50 to 200; about 100 to 200; and about 175 to 240), as measured in a static gel strength assay. Gel strengths can be measured in the presence and absence of a cation source, such as a calcium source.

A fluid ingestible composition can exhibit a viscosity in the range of from about 15 to about 100 cPs, or any value therebetween, at a shear rate of about 10^{-S}, e.g., about 17 to about 24; about 20 to about 25; about 50 to 100, about 25 to 75, about 20 to 80, or about 15 to about 20 cPs. Viscosity can be measured by those skilled in the art, e.g., by measuring flow curves of solutions with increasing shear rate using a double gap concentric cyclinder fixture (e.g., with a Parr Physica Rheometer).

A fluid ingestible composition can include a cation sequestrant, e.g., to prevent premature gellation of the anionic soluble fibers. A cation sequestrant can be selected from EDTA and its salts, EGTA and its salts, sodium citrate, sodium hexametaphosphate, sodium acid pyrophosphate, trisodium phosphate anhydrous, tetrasodium pyrophosphate, sodium tripolyphosphate, disodium phosphate, sodium carbonate, and potassium citrate. A cation sequestrant can be from about 0.001% to about 0.3% by weight of the ingestible composition. Thus, for example, EDTA can be used at about 0.0015% to about 0.002% by weight of the ingestible composition and sodium citrate at about 0.230% to about 0.260% (e.g., 0.250%) by weight of the ingestible composition.

A fluid ingestible composition can include a juice or juice concentrate and optional flavorants and/or colorants. Juices for use include fruit juices such as apple, grape, raspberry, blueberry, cherry, pear, orange, melon, plum, lemon, lime, kiwi, passionfruit, blackberry, peach, mango, guava, pineapple, grapefruit, and others known to those skilled in the art. Vegetable juices for use include tomato, spinach, wheatgrass, cucumber, carrot, peppers, beet, and others known to those skilled in the art.

The brix of the juice or juice concentrate can be in the range of from about 15 to about 85 degrees, such as about 25 to about 50 degrees, about 40 to about 50 degrees, about 15 to about 30 degrees, about 65 to about 75 degrees, or about 70 degrees. A liquid ingestible composition can have a final brix of about 2 to about 25 degrees, e.g., about 5, about 10, about 12, about 15, about 20, about 2.5, about 3, about 3.5, about 3.8, about 4, or about 4.5.

Flavorants can be included depending on the desired final flavor, and include flavors such as kiwi, passionfruit, pineapple, coconut, lime, creamy shake, peach, pink grapefruit, peach grapefruit, pina colada, grape, banana, chocolate, vanilla, cinnamon, apple, orange, lemon, cherry, berry, blueberry, blackberry, apple, strawberry, raspberry, melon(s), coffee, and others, available from David Michael, Givaudan, Duckworth, and other sources.

Colorants can also be included depending on the final color to be achieved, in amounts quantum satis that can be determined by one having ordinary skill in the art.

Rapid gelling occurs when soluble anionic fibers, such as alginate or pectin, are mixed with soluble calcium sources, particularly the calcium salts of organic acids such as lactic or citric acid. For beverage products, this reactivity prevents the administration of soluble anionic fiber and a highly soluble calcium source in the same beverage. In the present invention, this problem is overcome by administering the soluble anionic fiber and the soluble calcium source in different product components.

Solids

At least one anionic soluble fiber can be present in a solid ingestible composition in any form or in any mixtures of forms. A form can be a processed, unprocessed, or both. Processed forms include extruded forms, spray-dried forms, roll-dried forms, or dry-blended forms. For example, a snack bar can include at least anionic soluble anionic fiber present as an extruded food product (e.g., a crispy), at least one anionic soluble fiber in an unextruded form (e.g., as part of the bar), or both.

An extruded food product can be cold- or hot-extruded and can assume any type of extruded form, including without limitation, a bar, cookie, bagel, crispy, puff, curl, crunch, ball, flake, square, nugget, and snack chip. In some cases, an extruded food product is in bar form, such as a snack bar or granola bar. In some cases, an extruded food product is in cookie form. In other cases, an extruded food product is in a form such as a crispy, puff, flake, curl, ball, crunch, nugget, chip, square, chip, or nugget. Such extruded food products can be eaten as is, e.g., cookies, bars, chips, and crispies (as a breakfast cereal) or can be incorporated into a solid ingestible composition, e.g., crispies incorporated into snack bars.

A solid form may also be a lollipop or a lolly that is made of hardened, flavored sugar mounted on a stick and intended for sucking or licking. One form of lollipop has a soft-chewy filling in the center of the hardened sugar. The soft center filling may be a gum, fudge, toffee, caramel, jam, jelly or any other soft-chewy filling known in the art. The at least one multivalent cation may be in the soft-chewy center or the harnend sugar. Likewise, at least fiber may be in the soft-chewy center or the harnend sugar. A hard candy filled with a soft center filling is another embodiment of the present invention. This embodiment is similar to the lollipop, except it is not mounted on a stick. The soft-chewy filling may be in the center or swirled or layered with the hard sugar confection.

A cookie can include at least one soluble anionic fiber in an unprocessed form or in a processed (e.g., extruded) form. A snack chip can include at least one soluble anionic fiber in extruded form or in spray-dried form, or both, e.g., an extruded anionic soluble fiber-containing chip having at least one anionic soluble fiber spray-dried on the chip.

A solid ingestible composition can include optional additions such as frostings, coatings, drizzles, chips, chunks, swirls, or layers. Such optional additions can include at least one protected cation, at least one anionic soluble fiber, or both.

Solid ingestible compositions can provide any amount from about 0.5 g to about 10 g total anionic soluble fiber per serving, e.g., about 0.5 g to about 5 g, about 1 g to about 6 g, about 3 g to about 7 g, about 5 g to about 9 g, or about 4 g to about 6 g. For example, in some cases, about 1 g, about 2 g, about 3 g, about 4 g, about 5 g, about 6 g, about 7 g, about 8 g, or about 9 g of anionic soluble fiber per serving can be provided.

A solid ingestible composition can include at least one anionic soluble fiber at a total weight percent of the ingestible composition of from about 4% to about 50% or any value therebetween. For example, a solid ingestible composition can include at least one anionic soluble fiber of from about 4% to about 10% by weight; or about 5% to about 15% by weight; or about 10% to about 20% by weight; or about 20% to about 30% by weight; or about 30% to about 40% by weight; or about 40% to about 50% by weight.

An extruded food product can be from about 0% to 100% by weight of an ingestible composition, or any value therebetween (about 1% to about 5%; about 5% to about 10%; about 10% to about 20%; about 20% to about 40%; about 30% to about 42%; about 35% to about 41%; about 37% to about 42%; about 42% to about 46%; about 30% to about 35%; about 40% to about 50%; about 50% to about 60%; about 60% to about 70%; about 70% to about 80%; about 80% to about 90%; about 90% to about 95%; about 98%; or about 99%). For example, an extruded bar, cookie, or chip can be about 80% to about 100% by weight of an ingestible composition or any value therebetween.

Alternatively, an ingestible composition can include about 30% to about 55% by weight of an extruded food product or any value therebetween, e.g., about 32%, about 33%, about 34%, about 35%, about 36%, about 37%, 3 about 8%, about 39%, about 40%, about 42%, about 45%, about 48%, about 50%, about 52%, or about 54% by weight of an extruded food product. For example, a snack bar composition can include extruded crispies in an amount of from about 32% to about 46% by weight of the snack bar.

An ingestible composition or extruded food product can include one or more of the following: cocoa, including flavonols, and oils derived from animal or vegetable sources, e.g., soybean oil, canola oil, corn oil, safflower oil, sunflower oil, etc. For example, an extruded food product can include cocoa or oils in an amount of about 3% to about 10% (e.g., about 3% to about 6%, about 4% to about 6%, about 5%, about 6%, about 7%, or about 4% to about 8%) by weight of the extruded food product.

Crispies

An extruded food product for inclusion in an ingestible composition can be a crispy. For example, crispies that include one or more alginates and/or pectins in a total amount of about 30% to about 35% by weight of the crispy can be included in a snack bar in an amount of about 32% to about 45% by weight of the snack bar. Crispies can be prepared using a fiber blend as described herein. Crispies can also include, among other things, about 52% to about 58% by weight of the crispy one or more of a rice flour, corn meal, and/or corn cone; and about 2% to about 10% by weight of the crispy of a protein isolate. Crispies can be prepared using methods known to those having ordinary skill in the art, including cold and hot extrusion techniques.

The soluble anionic fiber can be provided in one beverage component, and a protected cation provided in a second beverage component. The first component and the second component are provided separately to the user in a bottle or cup, and the user consumes the two components concurrently or sequentially.

The soluble anionic fiber may be delivered in a beverage component and a protected cation may be provided separately in a solid edible component. The liquid fiber component and the solid cation containing component are consumed concurrently or sequentially.

The soluble anionic fiber component may be provided in a solid edible component, and the protected cation may be provided separately in a liquid component. The liquid protected cation—containing component and the solid fiber-containing component are consumed concurrently or sequentially.

The invention, the soluble anionic fiber component and the protected cation may both be provided in solid edible components. The components may be provided in the form of separate items for consumption, or both components may be combined in a single solid form for consumption. This single solid form may contain the soluble anionic fiber in one phase, such as a layer or filling, and the protected cation may be provided in a separate phase, such as a layer or filling. Alternatively, the fiber and protected cation may be intimately mixed in the same solid form.

The ingestible composition of the present invention can be provided in any package, such as enclosed in a wrapper or included in a container. An ingestible composition can be included in an article of manufacture. An article of manufacture that includes an ingestible composition described herein can include auxiliary items such as straws, napkins, labels, packaging, utensils, etc.

An article of manufacture can include a source of at least one protected cation. For example, a source of at least one protected cation can be provided as a liquid, e.g., as a beverage to be consumed before, during, or after ingestion of the ingestible composition. In other cases, at least one protected cation can be provided in a solid or gel form. For example, a source of at least one protected cation can be provided in, e.g., a jelly, jam, dip, or pudding, to be eaten before, during, or after ingestion of the ingestible composition. Thus, in some embodiments, an article of manufacture that includes a cookie or bar solid ingestible composition can also include a dip comprising a source of at least one protected cation, e.g., into which to dip the cookie or bar solid ingestible composition.

Also provided are articles of manufacture that include a fluid ingestible composition. For example, a fluid ingestible composition can be provided in a container. Supplementary items such as straws, packaging, labels, etc. can also be included. Alternatively, the soluble anionic fiber may be included in a beverage and the cation may be provided inside, outside or both of a straw or stirring stick. In some cases, at least one protected cation, as described below, can be included in an article of manufacture. For example, an article of manufacture can include a fluid ingestible composition in one container, and a source of cations in another container. Two or more containers may be attached to one another.

Methods of Reducing Caloric Consumption

An anionic soluble fiber (such as alginate and pectin) is administered concurrently with a protected cation source such as a water-soluble calcium salt to reduce food intake. Continued use of these compositions by individuals in need of weight loss will result in a cumulative decrease in caloric consumption, which will result in weight loss or diminished weight gain. Although not wishing to be bound by theory, the inventors hypothesize that the cations cross link the carboxylate groups on the fiber molecules, resulting in the formation of highly viscous or gelled materials. This gelling effect increases the viscosity of the gastric and intestinal contents, slowing gastric emptying, and also slowing the rate of macro-nutrient, e.g., glucose, amino acids, fatty acids, and the like, absorption. These physiological effects prolong the period of nutrient absorption after a meal, and therefore prolong the period during which the individual experiences an absence of hunger. The increased viscosity of the gastrointestinal contents, as a result of the slowed nutrient absorption, also causes a distal shift in the location of nutrient absorption. This distal shift in absorption may trigger the so-called “ileal brake”, and the distal shift may also cause in increase in the production of satiety hormones such as GLP-1 and PYY

Provided herein are methods employing the ingestible compositions described herein. For example, a method of facilitating satiety and/or satiation in an animal is provided. The method can include orally administering an ingestible composition to an animal. An animal can be any animal, including a human, monkey, mouse, rat, snake, cat, dog, pig, cow, sheep, bird, or horse. Oral administration can include providing the ingestible combination either alone or in combination with other meal items. Oral administration can include co-administering, either before, after, or during administration of the ingestible composition, a source of at least one protected cation, such as, calcium or a sequestered source of calcium, as described herein. At least one protected cation can be administered within about a four hour time window flanking the administration of the ingestible composition. For example, a protected source of calcium, such as a calcium lactate lipid particle, can be orally administered to an animal immediately after the animal has ingested a liquid ingestible composition as provided herein. Satiety and/or satiation can be evaluated using consumer surveys (e.g., for humans) that can demonstrate a statistically significant measure of increased satiation and/or satiety.

Alternatively, data from paired animal sets showing a statistically significant reduction in total caloric intake or food intake in the animals administered the ingestible compositions can be used as a measure of facilitating satiety and/or satiation.

As indicated previously, the ingestible compositions provide herein can hydrate and gel in the stomach and/or small intestine, leading to increased viscosity in the stomach and/or small intestine after ingestion. Accordingly, provided herein are methods for increasing the viscosity of stomach and/or small intestine contents, which include administering an ingestible composition to an animal. An animal can be any animal, as described above, and administration can be as described previously. Viscosity of stomach contents can be measured by any method known to those having ordinary skill in the art, including endoscopic techniques, imaging techniques (e.g., MRI), or in vivo or ex vivo viscosity measurements in e.g., control and treated animals.

Also provided are methods for promoting weight loss by administering an ingestible composition as provided herein to an animal. Administration can be as described previously. The amount and duration of such administration will depend on the individual's weight loss needs and health status, and can be evaluated by those having ordinary skill in the art. The animal's weight loss can be measured over time to determine if weight loss is occurring. Weight loss can be compared to a control animal not administered the ingestible composition.

The exact amounts of soluble anionic fiber and protected cation to be administered will depend on the weight, age, sex, and physiologic state of the individual to be treated, and the magnitude of the desired effect. The specific amounts of soluble anionic fiber and protected cation needed for an individual can readily be determined by experience of the individual or the healthcare professional treating the individual.

The following examples are representative of the invention, and are not 5 intended to be limiting to the scope of the invention.

### EXAMPLES

### Example 1

An anionic fiber beverage of the following composition was prepared:

| Ingredient | Weight % |
|---|---|
| Water | 95.470 |
| Trisodium citrate dehydrate | 0.250 |
| LBA alginate (ISP) | 0.640 |
| GHB alginate (ISP) | 0.550 |
| USP-L200 pectin (Kelco) | 0.200 |
| Apple juice concentrate | 2.300 |
| EDTA | 0.200 |
| Sucralose (Tate & Lyle) | 0.011 |
| Malic acid | 0.200 |
| Red 40, 10% solution | 0.001 |
| Flavor | 0.380 |
| Total | 100.000 |

Enteric coated tablets of calcium lactate pentahydrate were prepared as follows:

A mixture containing 88.5% calcium lactate pentahydrate, 11.0% Methocel K100M, and 0.5% stearic acid is compressed on a Manesty Beta tablet press into small cylindrical tablets that were about 2.5 mm in length and about 2 mm in 15 diameter. The compression force was about 2.78 kN. The final weight of the individual tablets was about 10 mg.

An enteric coating (Acryl-ESE 20% TS suspension) was applied in a coating pan apparatus. The weight gain in the coating process was 8%. The calcium content of these coated tablets was about 10.6%.

Testing of the coated tablets in 0.1 N HCI indicated the effectiveness of the enteric coating; only about 19% of the calcium was released after 2 hours exposure to 0.1 N HCI. Dissolution of the enteric-coated tablets was rapid at neutral pH, with visible disintegration occurred after about 5 minutes. Control calcium tablets have identical cores that did not received an enteric coating.

Pig Fistula Model:

Fully grown female Yucatan minipigs (Charles River Laboratories, Wilmington, Mass.), weighing about 90 kg, were fitted with indwelling silicone rubber sample ports (Omni Technologies, Inc., Greendale, Ind.) implanted in a surgically created dermal fistula at the ileocecal junction. The sample ports were sealed by a removable cap. These ports permitted removal of samples of digesta as it passed from the ileum to the cecum. Additional details of this procedure are presented in B. Greenwood van-Meerveld et al., Comparison of Effects on Colonic Motility and Stool Characteristics Associated with Feeding Olestra and Wheat Bran to Ambulatory Mini-Pigs, Digestive Diseases and Sciences 44:1282-7 (1999), which is incorporated herein by reference.

Samples of digesta were collected in sealed plastic containers. Viscosity of the digesta was measured with a Stevens QTS Texture Analyzer (Brookfield Engineering, Inc., Middleboro, Mass.). This instrument measured the relative viscosity of digesta by a back extrusion technique. The instrument had a stage plate, a 60 cm vertical tower, a mobile beam and a beam heat that contained a load-cell. During back extrusion, the beam descended at a constant rate, and the force required to back extrude the sample was recorded over time. The sample containers were about 5 cm deep spherical aluminum cups with an internal diameter of about 2.0 cm. The volume of the cup was about 20 ml. The spherical probe included a 1.9 cm TEFLON ball mounted on a 2 mm threaded rod which was attached to the mobile beam. The diameters of the sample cup and probe allow for a wide range of viscosity (liquid to solid digesta) to be measured without approaching the maximum capacity of the rheometer (25 kg/peak force). During each test, the beam thrusted the probe into the test sample at a constant rate (12 cm/second) for a 2 cm stroke, forcing the sample to back-extrude around the equatorial region of the probe. The peak force for back extrusion at a controlled stroke rate was proportional to the viscosity of the sample. For each data point presented, 2-6 samples from each pig at each time-point were tested and the mean peak force was calculated and recorded.

Pig Fistula Experimental Procedure:

Three Yucatan minipigs were housed in individual stainless steel pens in windowless room maintained on a cycle of 12 hours of light and 12 hours of dark. They were conditioned to consume low fiber chow (Laboratory Mini-Pig Diet 5L80, PMI Nutritional International, Brentwood, Mo.). This chow contained about 5.3% fiber. The pigs were fed once each day, in the morning. Water was provided ad lib throughout the day.

Samples were taken from the ileal sample port immediately after feeding, and then at about 30 minute intervals for about 300 minutes. The volume of sample collected was about 50 to 130 ml. All samples were assayed for viscosity within 30 minutes after collection.

Low fiber control consisted of low fiber chow without fiber supplementation.

On days on which the calcium particles discussed above were tested, or the fiber beverage only controls, about 50 g of the daily ration of pig chow was set aside, and the remainder was placed into the feeding bowl. With constant stirring, 16 ounces of the beverage described above were mixed into the chow. After the chow was uniformly moistened, the 50 g of withheld chow was sprinkled onto the surface, creating a dry layer. The enteric coated calcium tablets (7.64 g) described above were sprinkled on top of the dry chow. This procedure was intended to prevent hydration and dissolution of the enteric coating prior to consumption of the chow and tablets. Three pigs were each fed this mixture on each of three separate days (9 determinations). Controls consisted of chow to which only the beverage was added, and chow to which the uncoated calcium tablets were added in the same manner as the enteric coated tablets. The amount of uncoated calcium tablets added was adjusted to provide the same amount of elemental calcium as was present in the enteric coated tablets. Again, three pigs were fed each on these controls on each of separate days.

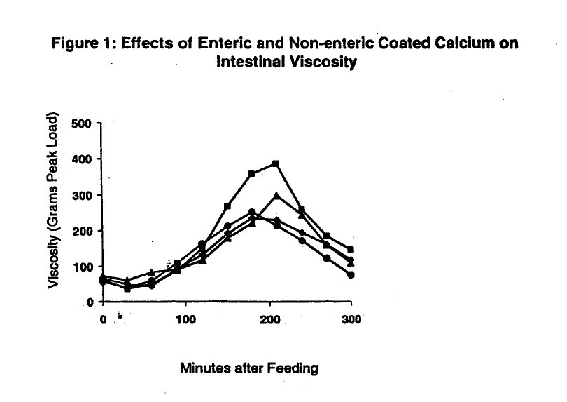

FIG. 1 presents results from this work. The results of the fiber beverage alone, as well as in combination with the enteric coated and the uncoated tablets, are compared with a sample of low fiber chow without fiber beverage. The presence of the enteric coated calcium caused a large increase in viscosity of the ileal effluent beginning at about 150 minutes after food consumption. While not wishing to be bound by theory, the inventors believe that the relatively small size of the enteric coated calcium particles allowed them to readily pass from the stomach to the intestine along with the other contents of the meal (in contrast to particles larger than about 3-5 mm diameter, which tend to be retained in the stomach until the end of meal emptying). After reaching the intestine, the enteric coating dissolved, releasing calcium from the tablets. In the neutral pH environment of the intestine, the carboxylate groups on the alginate molecules were fully charged. This facilitated ready complexing of the alginate carboxylate groups by the liberated calcium. As a result of the multivalent nature of the calcium ions, calcium was able to crosslink two carboxylate groups, thus forming intramolecular or intermolecular bridges with the alginate. This substantially increased the viscosity of the alginate, thus accounting for the enhanced viscosity noted in FIG. 1. A noteworthy observation is that although the calcium-alginate complexes probably formed in the most proximal areas of the small intestine, the resulting viscosity persisted to the distal ileum. This indicates that the enhanced viscosity produced was relatively stable despite the peristalsis and shearing that occur in the small intestine.

### Example 2

Calcium Composition:

Particles containing calcium lactate pentahydrate and a mixture of acyl glyceride lipids were obtained Balchem Corporation, Slate Hill, N.Y. The process for producing these particles is described generally by U.S. Pat. No. 6,797,191, which is incorporated herein by reference.

Greater than 98% of the mass of the particles include particles with a diameter from about 95 to about 1500 microns; wherein approximately 60% of the particles were between about 180 and about 425 microns. The remainder of the particles had a diameter of less than about 95 microns. The particles contained about 50% calcium lactate pentahydrate and about 50% lipid coating.

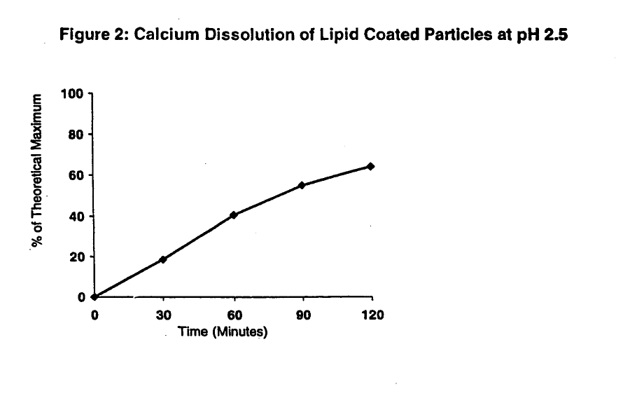

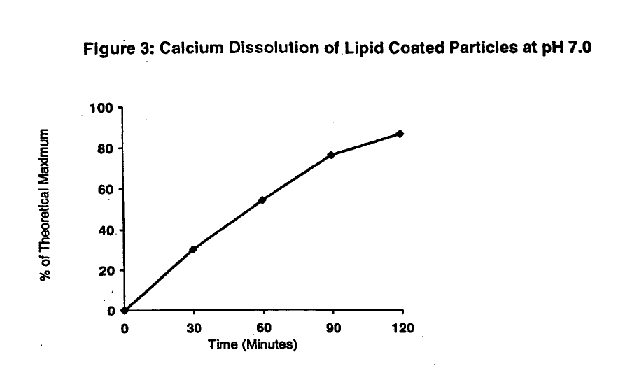

The final particles displayed enteric release properties when tested using the test described at United States Pharmacopeia 23^{rd} Edition, p. 1793, section 724, (1995). FIG. 2 presents the release of calcium from these particles under conditions simulative of the fed stomach (pH 2.5). FIG. 3 similarly presents the release of calcium from these particles under conditions simulative of the intestine (pH 7.2). As seen in the data, the calcium was relatively protected from dissolution under acidic conditions, but dissolution or solubilization proceeded much more rapidly under the neutral conditions.

The fiber containing beverage from Example 1 was used in this example.

Male Sprague-Dawley rats weighing 200-250 g were used. Rats were housed in a closed facility with a 12 hour day-night light cycle. Rats were fasted for 24 hours prior to dosing, but were provided ad lib access to water. A stainless steel tube was used to gavage rats with 5 ml of test solutions. Rats were returned to cages after gavage. At 30 minutes after gavage, rats were asphyxiated with carbon dioxide, and the small intestine was clamped at each end and removed. The small intestine was additionally clamped at a position equal to one half of the length. Contents were removed from the proximal half by gently passing the intestine between the firmly clasped thumb and index finger of a gloved hand. Contents were expressed into a tared vial, and the weight of the collected sample was determined. Samples were held on ice until viscosity measurements were performed, which occurred within one hour of collection.

Viscosity was measured with a RVDV1 CP viscometer (Brookfield Engineering Laboratories, Middleboro, Mass.). The viscometer was equipped with a circulating water bath to maintain sample temperature at 370° C. A Brookfield CPE 42 cone and plate spindle apparatus was used to measure the sample viscosity.

The beverage described above was diluted 1:2 with purified water and held at room temperature.

A 10 ml aliquot of the diluted beverage was transferred into a plastic scintillation vial, and 30 mg of the calcium—containing lipid particles described in Example 2 were added. The sample was vortexed for approximately 20 seconds to obtain uniform dispersion of the particles, and a 5 ml aliquot was immediately removed with a syringe and administered to a rat by gavage.

Control samples administered to rats included 5 ml of purified water, and 5 ml of the 1:2 diluted beverage without added calcium particles that was similarly vortexed.

The following results were obtained for samples of intestinal contents:

| | Number of | Mean Viscosity (centipoises) +/− SEM at Indicated Spindle RPM | | | |
|---|---|---|---|---|---|
| Treatment | Animals | 2 | 4 | 10 | 20 |
| Water | 8 | 739.8 +/− 98.7 | 405.4 +/− 58.1 | 214.8 +/− 27.6 | 61.2 +/− 21.6 |
| 1:2 Beverage | 8 | 768.3 +/− 85.1 | 400.8 +/− 50.7 | 217.8 +/− 25.1 | 158.6 +/− 20.4 |
| 1:2 Beverage | 7 | 962.4 +/− 214.0 | 564.6 +/− 146 | 273.3 +/− 53.6 | 199.2 +/− 41.3 |
| with Calcium | | | | | |

The samples display strongly non-Newtonian behavior (that is, viscosity decreased with increasing shear); which is commonly observed with high molecular polymers in biological systems. The presence of the enteric calcium particles increased viscosity compared to the effects of the beverage alone. While not wishing to be held by theory, the inventors hypothesize that the calcium is released from the particles after the particles reached the neutral pH of the intestine and the free calcium cross-linked some of the carboxylate groups on the alginate polymers. This crossing created a more rigid matrix of greater apparent molecular weight, which increased the viscosity of the samples in comparison to the water controls or the diluted beverage without calcium.

### Example 3

Forty moderately overweight (body mass index of 25-30) male and female subjects are recruited. On separate days one week apart, the subjects report to the test location in late morning after having eaten a standardized breakfast before 9 AM. On one of the sample days, the subjects receive an active treatment (anionic fiber with enteric coated multivalent cation); on the other day, the subjects receive placebo (anionic fiber with uncoated multivalent cation). The treatment order is randomized, and neither the subject nor the investigator knows the identity of the samples.

The active treatment comprises drinking 16 ounces of the beverage described in Example 1, as well as consuming 7.64 g of the enteric-coated calcium particles described in Example 1. The calcium particles are included in size 00 gelatin capsules shells to facilitate swallowing, and avoid damage to the particles during mastication. The capsules are swallowed as the beverage is consumed. The control group receives the same beverage, but they receive an identical amount of calcium in the form of uncoated calcium particles from the same preparation as used for enteric coating. These particles are similarly enclosed in gelatin capsule shells.

Immediately after consuming the above treatments, the subjects receive a standard lunch providing 700 kcal. Just after completing lunch, the subjects complete visual analog scales related to satiety, appetite, and sense of amount of food they could eat. These visual analog scales are repeated every 30 minutes for five hours.

Analysis of the data from the visual analog scales indicates that the enteric-coated calcium treatment produces a greater and more prolonged sense of satiety than does the control treatment, and also produces a decreased appetite and decreased expectation of how much food could be consumed.

What is claimed is:

1. An ingestible composition to increase viscosity imparted to the digesta in an intestine the composition comprising
an effective amount of at least one soluble anionic fiber and
an effective amount of at least one protected cation to increase viscosity
imparted to the digesta in the intestine.

2. An ingestible composition of claim 1, wherein the protected cation is selected from the group consisting of calcium, magnesium, aluminum, manganese, iron, nickel, copper, zinc, strontium, barium, bismuth, chromium, vanadium, strontium, lanthanum, their salts and mixtures thereof.

3. An ingestible composition of claim 2, wherein the protected cation is calcium.

4. An ingestible composition of claim 3, wherein the calcium salts are selected from the group consisting of citrate, tartrate, malate, formate, lactate, gluconate, phosphate, carbonate, sulfate, chloride, acetate, propionate, butyrate, caprylate, valerate, fumarate, adipate, succinate, and mixtures thereof.

5. An ingestible composition of claim 1, wherein the anionic soluble fiber is selected from the group consisting of alginate, pectin, gellan, soluble fibers that contain carboxylate substituents, carrageenan, polygeenan, marine algae-derived polymers that contain sulfate substituents, and mixtures thereof.

6. An ingestible composition of claim 2, wherein the anionic soluble fiber is selected from the group consisting of alginate, pectin, gellan, soluble fibers that contain carboxylate substituents, carrageenan, polygeenan, marine algae-derived polymers that contain sulfate substituents, and mixtures thereof.

7. An ingestible composition of claim 3, wherein the anionic soluble fiber is selected from the group consisting of alginate, pectin, gellan, soluble fibers that contain carboxylate substituents, carrageenan, polygeenan, marine algae-derived polymers that contain sulfate substituents, and mixtures thereof.

8. An ingestible composition of claim 4, wherein the anionic soluble fiber is selected from the group consisting of alginate, pectin, gellan, soluble fibers that contain carboxylate substituents, carrageenan, polygeenan, marine algae-derived polymers that contain sulfate substituents, and mixtures thereof.

9. An ingestible composition of claim 1, wherein the soluble anionic fiber is in a solid or liquid form and the protected cation is in a solid form.

10. An ingestible composition of claim 1, wherein the enteric coated multivalent cation is calcium and anionic soluble fiber is alginate.

11. An ingestible composition of claim 10, wherein alginate and calcium are present in a weight ratio of from about 5:1 to about 40:1.

12. An ingestible composition of claim 11, wherein the weight ratio of alginate to calcium from about 7:1 to about 20:1.

13. The ingestible composition of claim 12, wherein the weight ratio of alginate to calcium is from about 10:1 to about 14: 1.

14. An ingestible composition of claim 1, wherein the protected cation is a coating selected from the group consisting of enteric coating, lipid coating, and sustained release coating.

15. An ingestible composition of claim 1 wherein the at least one soluble anionic fiber is a fiber blend.

16. An ingestible composition of claim 15, wherein the fiber blend comprises alginate and pectin.

17. An ingestible composition of claim 16, wherein the alginate and pectin are present in a ratio of total alginate to total pectin of from about 8:1 to about 5:1.

18. An ingestible composition of claim 17, wherein the alginate and pectin are present in a ratio of total alginate to total pectin of from about 7:1 to about 6.5:1.

19. An ingestible composition of claim 18, wherein the alginate and pectin are present in a ratio of total alginate to total pectin of about 6.2:1.

20. An ingestible composition comprising:
at least one anionic soluble fiber and
a protected neutral pH soluble calcium salt in solid form.

21. An ingestible composition comprising:
at least one anionic soluble fiber and
a cation containing lipid composition, the cation- containing lipid
composition comprising a neutral pH soluble multivalent cation salt or
complex, wherein the multivalent cation salt or complex is at least
partially coated with a composition of monoglycerides, diglycerides, or
triglycerides, or combinations thereof.

22. An ingestible composition of claim 21, wherein the cation-containing lipid composition is introduced into the ingestible composition as discrete particles.

23. An ingestible composition of claim 22, wherein the mean diameter of the cation-containing lipid composition particles is from about one micron to about 1000 microns.

24. An ingestible composition of claim 23, wherein the mean diameter of the cation-containing lipid composition particles is from about 200 microns to about 500 microns.

25. An ingestible composition of claim 21, wherein the effective amount anionic soluble fiber and the effective amount of cation-containing lipid composition are consumed within a time interval of less than about two hours.

26. An ingestible composition comprising:
at least one anionic soluble fiber and
a plurality of enteric coated cation particles.

27. An ingestible composition of claim 26, wherein the mean diameter of the enteric coated cation are from about 10 microns to about 5 mm.

28. An ingestible composition of claim 27, wherein the mean diameters of the enteric cation are about 50 microns to about 3 mm.

29. An ingestible composition of claim 28, wherein the mean diameters of the enteric coated cation are about 100 microns to about 2 mm.

30. An ingestible composition of claim 26, wherein the enteric coating comprises a compound selected from the group consisting of methacrylic acid copolymer, zein, chitosan and mixtures thereof.
